(19) United States
(12) Patent Application Publication
(10) Pub. No.: US 2010/0160944 A1
(43) Pub. Date: Jun. 24, 2010

(54) Thermally detachable embolic assemblies

(75) Inventors: Clifford Teoh, Los Altos, CA (US); John M. K. Daniel, Fremont, CA (US)

Correspondence Address: VISTA IP LAW GROUP LLP, 12930 Saratoga Avenue, Suite D-2, Saratoga, CA, 95070, US

(73) Assignee: Boston Scientific Scimed, Inc.

(21) Appl. No.: 12/589,756

(22) Filed: Oct. 28, 2009

Related U.S. Application Data
(60) Provisional application No. 61/203,603, filed on Dec. 24, 2008.

(51) Int. Cl. A61M 29/00; A61F 2/06; C09J 5/00

(52) U.S. Cl. 606/191; 623/1.11; 156/322; 606/194

(57) ABSTRACT

Disclosed herein are vaso-occlusive assemblies for deploying implantable devices into the vasculature of a patient. More particularly, disclosed herein are vaso-occlusive assemblies comprising at least one thermally detachable polymer structure permanently attached to the implantable device and non-permanently attached to the delivery device prior to deployment. Also described are methods of making and using these assemblies.

### CROSS-REFERENCE TO RELATED APPLICATIONS

This application claims the benefit of U.S. provisional patent application No. 61/203,603 filed Dec. 24, 2008, the disclosure of which is incorporated by reference in its entirety for all purposes.

### FIELD OF THE INVENTION

Compositions and methods for repair of aneurysms are described. In particular, stretch-resistant vaso-occlusive devices are described, including stretch-resistant vaso-occlusive devices with flexible, articulating detachment junctions.

BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.).

In addition, coil designs including stretch-resistant members that run through the lumen of the helical vaso-occlusive coil have also been described. See, e.g., U.S. Pat. Nos. 5,582,619; 5,833,705; 5,853,418; 6,004,338; 6,013,084; 6,179,857; 6,193,728 and U.S. Patent Publication No. 20040002732. U.S. Patent Publication No. 20070239193 discloses stretch-resistant vaso-occlusive devices with distal anchor link structures.

U.S. Pat. Nos. 6,620,152; 6,425,893; 5,976,131 5,354,295; and 5,122,136, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint. Coil devices with polymeric detachment junctions have also been described. U.S. Pat. No. 6,743,251 describes detachment junctions that are severed by the application of low frequency of alternating current. U.S. Patent Publication No. 20060271097 discloses flexible electrolytically detachable junctions.

However, none of these documents describe vaso-occlusive devices as described herein or methods of making and using such devices.

### SUMMARY

Thus, this invention includes novel occlusive compositions as well as methods of using and making these compositions.

In one aspect, described herein is a vaso-occlusive device comprising: an implantable device (e.g., helical coil) having proximal and distal ends; and at least one meltable polymer structure attached to the proximal and/or distal end of the implantable device, wherein the meltable polymer structure extends from the proximal end of the implantable device. In certain embodiments, the implantable device comprises a lumen and the polymer structure extends at least partially through the lumen of implantable device.

Any of the vaso-occlusive devices described herein may further comprise one or more stretch-resistant members extending through the lumen of the implantable device. Furthermore, in any of the devices described herein the polymer structure may comprise at least one polymer selected form group consisting of polyethylene, polypropylene, PET, PLGA, and Nylon.

In another aspect, described herein is a vaso-occlusive assembly comprising any of the vaso-occlusive devices described herein; a delivery device; and wherein the at least one meltable polymer structure is permanently secured to the implantable device and non-permanently linked to the delivery device such that upon application of heat the polymer structure melts in the region linking it to the delivery device. In certain embodiments, the delivery device comprises a heater coil operably connected to a source of electrical energy. In any of the assemblies described herein, the delivery device may further comprise a delivery coil. Furthermore, in any of the assemblies described herein the meltable polymer structure may be secured directly to the implantable device.

In yet another aspect, described herein is a method of making a vaso-occlusive assembly as described herein, comprising the steps of (a) permanently securing the meltable polymer structure to the implantable device; and (b) non-permanently linking the meltable polymer structure to the delivery device. In certain embodiments, the meltable polymer is threaded or looped through an element of the delivery device.

In a still further aspect, provided herein is a method of at least partially occluding an aneurysm, the method comprising the steps of introducing a vaso-occlusive assembly as described herein into the aneurysm and detaching the implantable device by melting at least part of the polymer structure such that the implantable device is deployed into the aneurysm.

Furthermore, any of the assemblies or devices described herein may further include one or more additional components.

These and other embodiments of the subject invention will readily occur to those of skill in the art in light of the disclosure herein.

### BRIEF DESCRIPTION OF THE FIGURES

FIG. 1 is a side, partial cross-section view depicting an exemplary vaso-occlusive assembly as described herein.

FIG. 2 is a side, partial cross-section view depicting another exemplary vaso-occlusive assembly as described herein.

FIG. 3 is a side, partial cross-section view depicting an exemplary heater coil element.

FIG. 4 is a side view depicting yet another exemplary vaso-occlusive assembly as described herein employing the heater coil element of FIG. 3.

FIG. 5, panels A and B, depict partial side views of an exemplary heater coil and meltable polymer design and depict simple non-permanent attachment of the meltable polymer to the heat coil.

### DETAILED DESCRIPTION

Occlusive (e.g., embolic) assemblies and devices are described. The devices described herein find use in vascular and neurovascular indications and are particularly useful in treating aneurysms, for example in small diameter, curved or otherwise difficult to access vasculature, for example aneurysms, such as cerebral aneurysms. Methods of making and using these vaso-occlusive are also aspects of this invention.

The devices described herein include a thermally detachable structure (e.g., a polymeric or otherwise meltable structure) that maintains the coil in association with the delivery device prior to detachment. However, unlike previously described vaso-occlusive devices containing thermally detachable tethers, in the assemblies described herein, the thermally detachable structures are non-permanently attached to the delivery device. By “permanently” or “secured” means that the structure is permanently attached via bonding or some other method. “Non-permanent” attachment refers to an assembly in which the thermally detachable structure maintains a pre-deployment association between coil and delivery device by being threaded or looped through a part of the delivery device. Thus, in the devices described herein, the thermally detachable structures are non-permanently attached to the delivery device such that they are sufficiently within the vicinity of a heater element to sever the device from the delivery device upon the application of energy to the heater element.

Furthermore, unlike currently available designs, the assemblies described herein may also include flexible, articulating detachment junctions. As noted above, implantable devices may be conveniently detached from the deployment mechanism (e.g., pusher wire) by the application of electrical energy to heat a heater element which in turn sufficiently melts the tether structure at or near the selected detachment region. However, many available electrolytically detachable implants are inflexible in or near the detachment junction. The detachment junction is preferably thermally detachable.

In addition, because the detachable tether structures described herein are not fixedly (permanently) attached to the delivery device, they can also function as stretch-resistant members for the implantable device (e.g., coil).

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to a device comprising “a polymer” includes devices comprising of two or more polymers.

The vaso-occlusive assemblies described herein include an implantable device, for example a vaso-occlusive device that is associated with a delivery device via a thermally detachable structure. Typically, the thermally detachable structure comprises one or more polymers, including but not limited to, one or more polymer filaments, for example constructs comprising filamentous elements assembled by one or more operations including coiling, twisting, braiding, weaving or knitting of the filamentous elements.

The thermally detachable structures described herein may be selected from a wide variety of materials. One such example is a suture-type material. Synthetic and natural polymers, such as polyurethanes (including block copolymers with soft segments containing esters, ethers and carbonates), polyethers, polyamides (including nylon polymers and their derivatives), polyimides (including both thermosetting and thermoplastic materials), acrylates (including cyanoacrylates), epoxy adhesive materials (two part or one part epoxy-amine materials), olefins (including polymers and copolymers of ethylene, propylene butadiene, styrene, and thermoplastic olefin elastomers), fluoronated polymers (including polytetrafluoroethylene), polydimethyl siloxane-based polymers, cross-linked polymers, non-cross linked polymers, Rayon, cellulose, cellulose derivatives such nitrocellulose, natural rubbers, polyesters such as lactides, glycolides, trimethylene carbonate, caprolactone polymers and their copolymers, hydroxybutyrate and polyhydroxyvalerate and their copolymers, polyether esters such as polydioxinone, anhydrides such as polymers and copolymers of sebacic acid, hexadecandioic acid and other diacids, or orthoesters may be used.

Thus, the polymer structures described herein may include one or more absorbable (biodegradable) polymers and/or one or more non-absorbable polymers. The terms “absorbable” and “biodegradable” are used interchangeable to refer to any agent that, over time, is no longer identifiable at the site of application in the form it was injected, for example having been removed via degradation, metabolism, dissolving or any passive or active removal procedure. Non-limiting examples of absorbable proteins include synthetic and polysaccharide biodegradable hydrogels, collagen, elastin, fibrinogen, fibronectin, vitronectin, laminin and gelatin. Many of these materials are commercially available. Fibrin-containing compositions are commercially available, for example from Baxter. Collagen containing compositions are commercially available, for example from Cohesion Technologies, Inc., Palo Alto, Calif. Fibrinogen-containing compositions are described, for example, in U.S. Pat. Nos. 6,168,788 and 5,290,552. Mixtures, copolymers (both block and random) of these materials are also suitable.

Preferred biodegradable polymers include materials used as dissolvable suture materials, for instance polyglycolic and/or polylactic acids (PGLA) to encourage cell growth in the aneurysm after their introduction. Preferred non-biodegradable polymers include polyethylene teraphthalate (PET or Dacron), polypropylene, polytetraflouroethylene, or Nylon materials. Highly preferred are PET or PGLA.

The polymeric structure is preferably fixedly (permanently) attached to implantable device. The polymeric structure may be produced prior to attachment to the implantable device, for example by fabricating or injection-molding a structure of the desired shape and fixedly attaching the pre-formed structure to the implantable device. Alternatively, all or a part of the polymeric structure may be shaped after it is attached to the implantable device, for example if a polymer comprising one or more filaments is fixedly attached to the implantable device and, after attachment to the implantable device, the filaments are non-permanently secured to the delivery device, for example by threading or looping through a portion of the delivery device.

The implantable device may be made of a variety of materials (e.g., metal, polymer, etc.) and may assume a variety of structures. Thus, although depicted in the Figures described below as a coil, the implantable device may be of a variety of shapes or configuration includes, but not limited to, braids, knits, woven structures, tubes (e.g., perforated or slotted tubes), cables, injection-molded devices and the like. See, e.g., U.S. Pat. No. 6,533,801 and International Patent Publication WO 02/096273. The implantable device preferably changes shape upon deployment, for example change from a constrained linear form to a relaxed, three-dimensional (secondary) configuration. See, also, U.S. Pat. No. 6,280,457.

In a particularly preferred embodiment, the implantable device comprises at least one metal or alloy. Suitable metals and alloys for the implantable device include the Platinum Group metals, especially platinum, rhodium, palladium, rhenium, as well as tungsten, gold, silver, tantalum, and alloys of these metals. The implantable device may also comprise of any of a wide variety of stainless steels if some sacrifice of radio-opacity may be tolerated. Very desirable materials of construction, from a mechanical point of view, are materials that maintain their shape despite being subjected to high stress. Certain “super-elastic alloys” include nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” These are very sturdy alloys that will tolerate significant flexing without deformation even when used as a very small diameter wire. If a super-elastic alloy such as nitinol is used in any component of the device, the diameter of the wire may be significantly smaller than that used When the relatively more ductile platinum or platinum/tungsten alloy is used as the material of construction. These metals have significant radio-opacity and in their alloys may be tailored to accomplish an appropriate blend of flexibility and stiffness. They are also largely biologically inert. In a preferred embodiment, the implantable device comprises a metal wire wound into a primary helical shape. The implantable device may be, but is not necessarily, subjected to a heating step to set the wire into the primary shape. The diameter of the wire typically making up the coils is often in a range of 0.0005 and 0.050 inches, preferably between about 0.001 and about 0.004 inches in diameter.

Methods of making vaso-occlusive coils having a linear helical shape and/or a different three-dimensional (secondary) configuration are known in the art and described in detail in the documents cited above, for example in U.S. Pat. No. 6,280,457. Thus, it is further within the scope of this invention that the vaso-occlusive device as a whole or elements thereof comprise secondary shapes or structures that differ from the linear coil shapes depicted in the Figures, for examples, spheres, ellipses, spirals, ovoids, figure-8 shapes, etc. The devices described herein may be self-forming in that they assume the secondary configuration upon deployment into an aneurysm. Alternatively, the devices may assume their secondary configurations under certain conditions (e.g., change in temperature, application of energy, etc.).

The thermally detachable structures are typically secured to the implantable device. They may be secured at the proximal and/or distal ends of the implantable device or may be secured at multiple locations along the implantable device. In certain embodiments, the thermally detachable structures extend partially or fully through the interior (e.g., lumen) of the implantable device. In other embodiments, the thermally detachable structures extend from the proximal end of the implantable device.

The thermally detachable structures may be secured to the implantable device in any fashion. For example, the polymeric structures may be wound around the implantable device or, alternatively, may be shaped into a tubular sheath that surrounds the implantable device. The polymer component may adhere to the implantable device in one or more locations, for example by heating (melting) of the polymer or by use of adhesives (e.g., EVA) to the polymer or to the implantable device), heat setting so as to shrink the polymer(s) onto the implantable device, or by other suitable means.

It will be apparent that the process used to attach the polymer to the implantable device will depend on the nature of the polymer. For example, it will be preferable not to heat certain polymers (e.g., PGLA) as heating may cause degradation of PGLA. Furthermore, the polymeric component may be added to the implantable device before or after the implantable device is shaped into a primary and/or secondary configuration.

As noted above, the detachable structure(s) are preferably not fixedly attached to the delivery device. Rather, these structures are associated with the delivery device such that, prior to deployment by melting of these structures, they link the implantable device to the delivery mechanism. Such non-permanent linkage may be achieved in a variety of ways, for example, by threading or looping the detachable structures through one or more portions of the delivery device.

Depicted in the Figures are exemplary embodiments of the present invention in which the implantable device is depicted as a helically wound metallic coil. It will be appreciated that the drawings are for purposes of illustration only and that other implantable devices can be used in place of embolic coils, for example, stents, filters, and the like. Furthermore, although depicted in the Figures as embolic coils, the embolic devices may be of a variety of shapes or configuration including, but not limited to, open and/or closed pitch helically wound coils, braids, wires, knits, woven structures, tubes (e.g., perforated or slotted tubes), injection-molded devices and the like. See, e.g., U.S. Pat. No. 6,533,801 and International Patent Publication WO 02/096273. It will also be appreciated that the devices and assemblies can have various configurations.

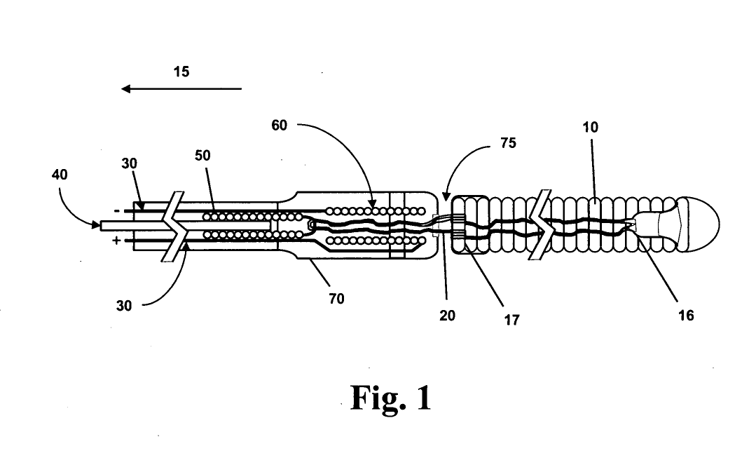

FIG. 1 is a schematic depicting a side, partial cross-section view of an exemplary assembly as described herein prior to deployment in which the thermally detachable structure extends through the lumen of implantable device. The exemplary assembly comprises a helically wound implantable device 10 with a meltable polymeric structure 20 attached to the distal end 16 of the implantable device 10 either directly or by a structure (e.g., ball cap) extending into the lumen of the implantable device 10. In this exemplary embodiment, the meltable polymeric structure is also attached to the proximal end of the coil, for example by looping it 17 around one or more winds of the proximal end of the implantable coil 10 as shown. In this embodiment, the meltable polymer structure 20 also serves as a stretch-resistant function in that it remains attached to the proximal and distal ends of the implantable device after deployment to keep the implantable device from over-stretching.

Also shown in FIG. 1 is the delivery device which includes electrodes 30, core (pusher) wire 40, delivery coil 50 and heater coil 60. The polymer structure 20 is secured to the proximal and distal ends of the implantable device 10 and to the distal region of the detachment region 75. The delivery device also includes a catheter (or sheath) 70 that can surround one or more elements of the delivery device. Also shown is detachment region 75 that is detached upon melting of the polymer 20 by the heater coil 60 upon application of electrical energy to heat the heater coil 60. Arrow 15 shows that the proximal end of core wire 40 is attached to a power supply.

The meltable polymeric structure 20 is also non-permanently attached to the delivery device. As shown in FIG. 1, the polymeric structure 20 is threaded through a loop on the distal end of the delivery coil 50. It will be apparent that the polymeric structure 20 can be non-permanently attached to any element of the delivery device, including, for example, the delivery coil 50 and/or the heater element 60.

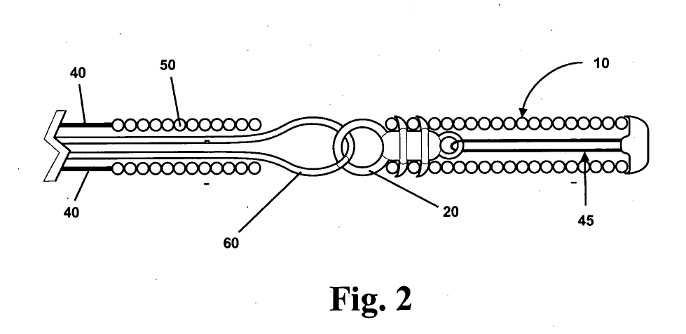

FIG. 2 shows another exemplary assembly in which the meltable polymeric structure 20 that is attached to the implantable device 10 at the proximal end of the coil. The polymeric structure 20 forms a loop that extends from the proximal end of the implantable device 10 and loops around a heater wire loop 60. Also shown are delivery coil 50 and pusher wires 40. Also shown in FIG. 2 is a stretch-resistant member 45 extending through the lumen of the implantable device 10 and is attached to both the meltable polymer structure 20 and the distal end of the implantable device 20 to provide stretch-resistant function upon detachment from the delivery device.

The meltable polymeric structure 20 may be secured directly at or near the proximal end of the implantable device 10. Alternatively, as shown in FIG. 2, the meltable polymeric structure may be integral to a structure that extends into the lumen of the device and/or attached to a structure that extends into the implantable device.

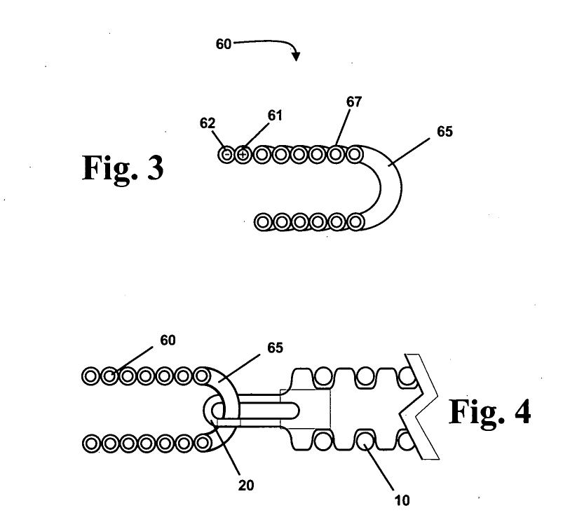

FIG. 3 shows an exemplary embodiment of a heater coil 60 which is created by winding a single wire into a coil forming a distal loop 65 and both positive 61 and negative 62 ends of the electrodes of the heater coil 60 towards the proximal end. The electrodes are preferably electrically insulated from each other by a layer of insulating material 67.

FIG. 4 shows the heater coil of FIG. 3 in an assembly as described herein. As shown, the loop 65 at the distal end of heater coil 60 contacts (but is not fixedly attached to) the meltable polymer structure 20 that is fixedly attached to the implantable device 10.

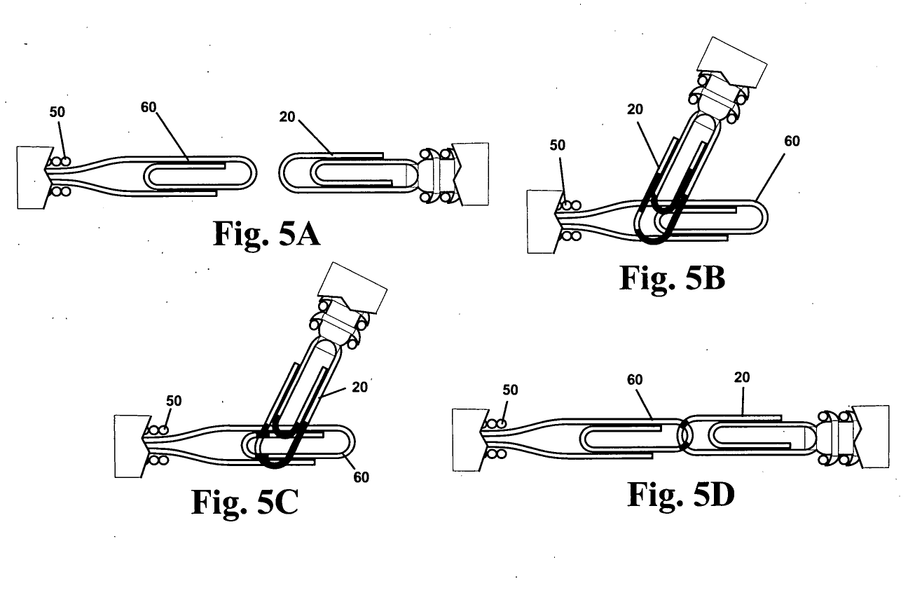

FIG. 5 shows another exemplary design in which the heater coil 60 and meltable polymer structures 20 are shaped into paper clip-like structures which can be easily attached to one another as with paper clips. Also shown is delivery coil 50. FIG. 5A shows paper clip-shaped heater coil 60 and paper clip-shaped meltable polymer structure 20 prior to attachment. The paper clip shaped part of the meltable polymer structure 20 extends proximally from a structure that is designed to secure the structure to the proximal end of an implantable device. FIG. 5B shows the first step of attachment of the paper clip shapes heater coil 60 and meltable polymer structure 20. As with paper clip attachment, the proximal loop of the meltable structure 20 is aligned with the proximal loop of the heater coil 60 for attachment. FIG. 5C shows the step of engaging the proximal loop of the meltable structure 20 with the proximal loop of the heater coil 60. Once engaged, the loops are intertwined by pulling the meltable polymer structure 20 distally until the proximal polymer structure 20 loop is looped around the distal loop of the heater coil 60. See, FIG. 5D.

As noted above, vaso-occlusive devices as described herein are conveniently detached from the deployment mechanism (e.g., pusher wire) by the application of electrical energy, which dissolves a suitable material at the selected detachment junction. The present invention also relates to flexible detachment junctions, which result in reduced catheter kickback and more efficient deployment. In particular, flexibility at the detachment zone may be imparted by attaching the polymer to the detachment junction in such a way that the stretch-resistant member is free to pivot with respect to the pusher wire.

One or more of the components of the devices described herein (e.g., polymer structure, implantable device) may also comprise additional components (described in further detail below), such as co-solvents, plasticizers, radio-opaque materials (e.g., metals such as tantalum, gold or platinum), coalescing solvents, bioactive agents, antimicrobial agents, antithrombogenic agents, antibiotics, pigments, radiopacifiers and/or ion conductors which may be coated using any suitable method or may be incorporated into the element(s) during production. In addition, lubricious materials (e.g., hydrophilic) materials may be used to coat one or more members of the device to help facilitate delivery. Cyanoacrylate resins (particularly n-butylcyanoacrylate), particular embolization materials such as microparticles of polyvinyl alcohol foam may also be introduced into the intended site after the inventive devices are in place. Furthermore, previously described fibrous braided and woven components (U.S. Pat. No. 5,522,822) may also be included, for example surrounding the implantable devices described herein.

One or more bioactive materials may also be included. See, e.g., co-owned U.S. Pat. No. 6,585,754 and WO 02/051460. The term “bioactive” refers to any agent that exhibits effects in vivo, for example a thrombotic agent, an anti-thrombotic agent (e.g., a water-soluble agent that inhibits thrombosis for a limited time period, described above), a therapeutic agent (e.g., chemotherapeutic agent) or the like. Non-limiting examples of bioactive materials include cytokines; extracellular matrix molecules (e.g., collagen); trace metals (e.g., copper); and other molecules that stabilize thrombus formation or inhibit clot lysis (e.g., proteins or functional fragments of proteins, including but not limited to Factor XIII, α₂-antiplasmin, plasminogen activator inhibitor-1 (PAI-1) or the like). Non-limiting examples of cytokines which may be used alone or in combination in the practice of the present invention include, basic fibroblast growth factor (bFGF), platelet derived growth factor (PDGF), vascular endothelial growth factor (VEGF), transforming growth factor beta (TGF-β) and the like. Cytokines, extracellular matrix molecules and thrombus stabilizing molecules (e.g., Factor XIII, PAI-1, etc.) are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly as the sequences of many of these molecules are also available, for example, from the GenBank database. Thus, it is intended that the invention include use of DNA or RNA encoding any of the bioactive molecules. Cells (e.g., fibroblasts, stem cells, etc.) can also be included. Such cells may be genetically modified. Furthermore, it is intended, although not always explicitly stated, that molecules having similar biological activity as wild-type or purified cytokines, extracellular matrix molecules and thrombus-stabilizing proteins (e.g., recombinantly produced or mutants thereof) and nucleic acid encoding these molecules are intended to be used within the spirit and scope of the invention. Further, the amount and concentration of liquid embolic and/or other bioactive materials useful in the practice of the invention can be readily determined by a skilled operator and it will be understood that any combination of materials, concentration or dosage can be used, so long as it is not harmful to the subject.

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced though an entry site in the vasculature. Typically, this would be through a femoral artery in the groin. Other entry sites sometimes chosen are found in the neck or in the arm and are in general well known by physicians who practice this type of medicine. Once the introducer is in place, a guiding catheter is then used to provide a safe passageway from the entry site to region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the vaso-occlusive device at the distal end, is advanced through the catheter.

Once the selected site has been reached, the vaso-occlusive device is detached by melting of the polymeric link. The implantable devices are held in place by gravity, shape, size, volume, magnetic field or combinations thereof.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device comprising:
an implantable device having proximal and distal ends;
at least one meltable polymer structure attached to the proximal and/or distal end of the implantable device, wherein the meltable polymer structure extends from the proximal end of the implantable device.

2. The vaso-occlusive device of claim 1, wherein the implantable device comprises a lumen and the polymer structure extends at least partially through the lumen of implantable device.

3. The vaso-occlusive device of claim 1, further comprising one or more stretch-resistant members extending through the lumen of the implantable device.

4. The vaso-occlusive device of claim 1, wherein the implantable device comprises a helical coil.

5. The vaso-occlusive device of claim 1, wherein the polymer structure at least one polymer selected form group consisting of polyethylene, polypropylene, PET, PLGA, and Nylon.

6. A vaso-occlusive assembly comprising
an implantable device according to claim 1;
a delivery device; and
wherein the at least one meltable polymer structure is permanently secured to the implantable device and non-permanently linked to the delivery device such that upon application of heat the polymer structure melts in the region linking it to the delivery device.

7. The vaso-occlusive assembly of claim 6, wherein the delivery device comprises a heater coil operably connected to a source of electrical energy.

8. The vaso-occlusive assembly of claim 6, wherein the delivery device further comprises a delivery coil.

9. The vaso-occlusive assembly of claim 7, wherein the delivery device further comprises a delivery coil.

10. The vaso-occlusive assembly of claim 6, wherein the meltable polymer structure is secured directly to the implantable device.

11. A method of making a vaso-occlusive assembly according to claim 6, comprising the steps of
(a) permanently securing the meltable polymer structure to the implantable device; and
(b) non-permanently linking the meltable polymer structure to the delivery device.

12. The method of claim 11, wherein the meltable polymer is threaded or looped through an element of the delivery device.

13. A method of at least partially occluding an aneurysm, the method comprising the steps of introducing a vaso-occlusive assembly according to claim 6 into the aneurysm and detaching the implantable device by melting at least part of the polymer structure such that the implantable device is deployed into the aneurysm.
